(19) United States
(12) Plant Patent Application Publication
(10) Pub. No.: US 2006/0041973 P1
(43) Pub. Date: Feb. 23, 2006

(54) Double impatiens plant named 'Cameo Cherry Red'

(75) Inventor: Xenia Cascante, Alajuela (CR)

Correspondence Address: THE WEBB LAW FIRM, P.C., 700 KOPPERS BUILDING, 436 SEVENTH AVENUE, PITTSBURGH, PA, 15219, US

(73) Assignee: Oglevee, Ltd., Connellsville, PA (US)

(21) Appl. No.: 11/209,339

(22) Filed: Aug. 23, 2005

Related U.S. Application Data
(60) Provisional application No. 60/603,582, filed on Aug. 23, 2004.

(51) Int. Cl. A01H 5/00

(52) U.S. Cl. PLT/317

(57) ABSTRACT

A new and distinct cultivar of Double Impatiens plant named ‘Cameo Cherry Red’, characterized by its compact, mounded and outwardly spreading plant habit; rapid growth rate; freely branching habit rounded green-colored leaves; dense foliage; freely flowering habit; fully double flower form; and large cherry red-colored flowers that are positioned above and beyond the foliage.

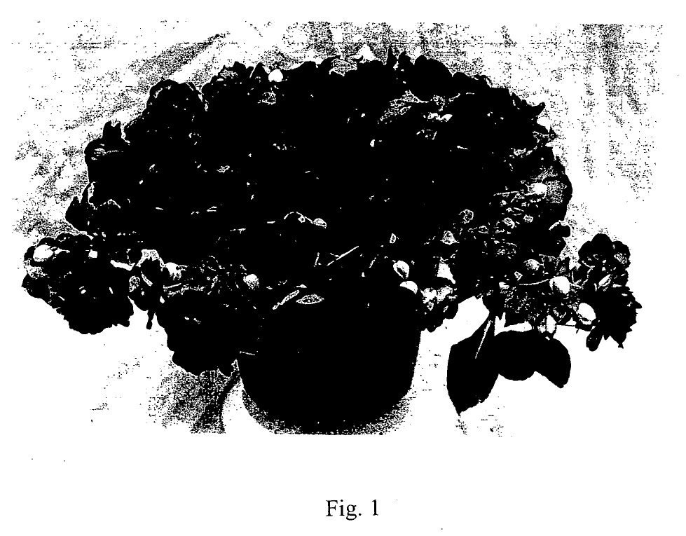

### CROSS-REFERENCE TO RELATED APPLICATIONS

This application claims the benefit of U.S. Provisional Plant Patent Application No. 60/603,582 filed Aug. 23, 2004, which is hereby incorporated by reference in its entirety.

### BOTANICAL CLASSIFICATION

Impatiens walleriana

### VARIETAL DENOMINATION

‘Cameo Cherry Red’

### BACKGROUND OF THE INVENTION

The present invention comprises a new and distinct cultivar of Impatiens walleriana, known by the cultivar name ‘Cameo Cherry Red’. The new variety is a product of a planned breeding program conducted in Cartago, Costa Rica. The purpose of the breeding program was to develop compact and mounded cultivars with fully double flowers, large flower size and cherry red flowers. The new variety originated from a cross made by the Inventor of two unnamed proprietary selections of Impatiens walleriana, not patented. Comparisons between the new variety and its parents cannot be made, as the parental varieties no longer exist. The cultivar ‘Cameo Cherry Red’ was discovered and selected as a flowering plant within the progeny of the stated cross in a controlled environment in Cartago, Costa Rica. Asexual reproduction of the new cultivar by terminal cuttings has shown that the unique features of the new variety are stable and reproduced true to type in successive propagations.

The following traits have been repeatedly observed and are determined to be the unique characteristics of ‘Cameo Cherry Red’. Characteristics that distinguish ‘Cameo Cherry Red’ as a new and distinct cultivar from others known to the breeder include:

1) Large flower size of 5 to 5.5 cm in diameter;

2) Compact, mounded, spreading growth habit;

3) Freely branching habit;

4) Rounded leaves with dense foliage;

5) Flower remains fully double even under stressful environmental conditions; and

6) Cherry red flowers are carried above the leaf canopy and face upward.

Plants of the new variety can be compared to plants of the Impatiens walleriana cultivar ‘Balolecher’, disclosed in U.S. Plant Pat. No. 13,668. In side-by-side comparisons conducted in Lompoc, Calif., plants of the new variety differed from plants of ‘Balolecher’ in the following characteristics:

1) Plants of the new variety had larger flowers than plants of ‘Balolecher’;

2) Plants of the new variety had flowers with a color of 52A, whereas plants of ‘Balolecher’ had flowers with a color of 53C;

3) Plants of the new variety had a stem color of 148C with a cast around the nodes of 184C, whereas plants of ‘Balolecher’ had a stem color of 147C with 184D on younger stems; and

4) Plants of the new variety had mature leaves with the underside having a color of 147C with a cast of 184D in areas, whereas plants of ‘Balolecher’ had mature leaves with the underside having a color of 148C with a cast of 184D in some areas.

Plants of the new variety can be compared to plants of the Impatiens walleriana cultivar ‘Cameo Scarlet’, disclosed in U.S. Plant Pat. No. 12,036. In side-by-side comparisons conducted in Lompoc, Calif., plants of the new variety differed from plants of ‘Cameo Scarlet’ in the following characteristics:

1) Plants of the new variety had larger flowers than the plants of ‘Cameo Scarlet’;

2) Plants of the new variety had flowers with a color of 52A, whereas the plants of ‘Cameo Scarlet’ had flowers with a color of 44B;

3) Plants of the new variety had longer leaves than plants of ‘Cameo Scarlet’; and

4) Plants of the new variety had mature leaves with the underside having a color of 147C with a cast of 184D in areas, whereas plants of ‘Cameo Scarlet’ had mature leaves with the underside having a color of 147B.

### BRIEF DESCRIPTION OF THE DRAWING

The accompanying colored photographic drawing illustrates the new variety with the colors being as nearly true as is possible to obtain in colored illustrations of this type.

### DESCRIPTION OF THE PLANT

The cultivar ‘Cameo Cherry Red’ has not been observed under all possible environmental conditions. The phenotype may vary somewhat with variations in environment such as temperature, day length and light intensity. However, there is no variance in genotype.

In the following description, color references are made to The Royal Horticultural Society Colour Chart, 1995 Edition, except where general terms of ordinary dictionary significance are used. Plants were grown under commercial practice during the late winter through early spring of 2003 in Lompoc, Calif., in a corrugated polycarbonate-covered greenhouse with 30% shade covering. During the production of the plants, day temperatures ranged from 21 to 24° C.; night temperatures ranged from 16 to 18° C.; and light levels ranged from 4000 to 5000 foot candles of light. Plants used in the following description were grown in 15 cm containers for about nine to ten weeks after planting rooted cuttings.
Propagation:
   Type cutting.—Terminal cuttings.
   Time to initiate roots.—Summer: About 10 days at 21° C. Winter: About 12 days at 21° C.
   Time to produce a rooted cutting.—Summer: About 21 days at 21° C. Winter: About 21 days at 21° C.
   Root description.—Fibrous, fine, white in color.
   Root habit.—Freely branching, dense.
Plant description:
   General appearance.—Compact, mounded and outwardly spreading. Appropriate for 10-15 cm pots and 20 to 25 cm hanging basket containers.
   Growth and branching habit.—Vigorous and freely-branching habit with at least 10 lateral branches developing per plant, dense growth. Pinching (removal of terminal apex) is usually not required.
   Crop time.—Relatively rapid growth rate. From planting one rooted cutting in a 10 cm. pot: About 5 to 6 weeks. From planting three rooted cuttings in a 25 cm. hanging basket container: About 8 to 10 weeks.
   Plant height at flowering.—About 18 to 20 cm.
   Plant width (spread).—About 28 cm.
   Lateral branch description.—Length: About 20 to 25 cm. Diameter: About 6 to 8 mm. Internode length: About 1 to 2.5 cm. Texture: Smooth, glabrous. Color: 148C with a cast of 184C near the nodes.
   Foliage description.—Arrangement: Alternate, simple; densely foliated. Length: About 4.5 to 5.0 cm. Width: About 2.5 to 3.0 cm. Shape: Ovate. Apex: Acute. Base: Acute. Margin: Serrated with ciliation. Texture, upper and lower surfaces: Glabrous. Venation pattern: Pinnate. Color: Developing leaves, upper surface: 146B. Developing leaves, lower surface: 146C. Fully expanded leaves, upper surface: 146A. Fully expanded leaves, lower surface: 147C with cast of 184D in some areas. Venation, upper surface: 147C. Venation, lower surface: 146B. Petiole length: About 10 to 15 mm. Petiole diameter: About 3 mm. Petiole texture upper and lower surface: Smooth, glabrous. Petiole color: 147A.
Flower description:
   Flower type and habit.—Numerous and consistently double cherry red flowers. Freely and continuously flowering. Flower buds open similar to a rose in fullness; flowers rounded. Usually 8 to 15 flowers and flower buds per lateral branch. Flowers are positioned above the foliage and typically face outward to upward. Flowers last about 7 to 10 days on the plant. Flowers not persistent. Flowers not fragrant.
   Flowering season.—Year-round under greenhouse conditions. In the garden, flowering from spring until fall. Flowering indeterminate and continuous.
   Flower diameter.—About 5 to 5.5 cm.
   Flower depth (height).—About 2 cm.
   Flower buds.—Rate of opening: From full color bud to full open flower, about 5 to 7 days is typical depending on temperature. Length: About 14 mm. Diameter: About 12 mm. Shape: Obovate. Color (just before opening): 51B.
   Petals.—Quantity/arrangement: About 35 to 40; imbricate. Length, outer petals: About 24 mm. Width, outer petals: About 22 mm. Shape: Ovate to cordate. Apex: Rounded with inner petals showing indentations. Base: Rounded to acute. Margin: Entire. Texture, upper and lower surfaces: Glabrous, smooth. Color: When opening, upper surface: 46A. When opening, lower surface: 43A. Fully opened, upper surface: 52A fading to 57B. Fully opened, lower surface: 43C.
   Peduncles.—Length: About 23 mm. Diameter: About 2 mm. Angle: About 45° to the lateral branch. Strength: Strong. Texture: Smooth, glabrous. Color: 147C.
Reproductive organs: Not applicable due to fully double flowers.
Fruit/Seed development: Fruit and seed development has not been observed.
Disease/Pest resistance: Plants of the new variety have not been observed to date to be resistant to pathogens and pests common to Impatiens.
Temperature tolerance: Plants of the new variety have been observed to be tolerant to rain, wind and temperatures ranging from 5 to 38° C.

I claim:

1. A new and distinct variety of Impatiens named ‘Cameo Cherry Red’ as herein described and illustrated.
